(19) United States
(12) Patent Application Publication
(10) Pub. No.: US 2024/0008738 A1
(43) Pub. Date: Jan. 11, 2024

(54) OCT APPARATUS

(71) Applicant: NIDEK CO., LTD., Gamagori-shi, Aichi (JP)

(72) Inventor: Yukihiro HIGUCHI, Gamagori-shi (JP)

(73) Assignee: NIDEK CO., LTD., Gamagori-shi, Aichi (JP)

(21) Appl. No.: 18/345,423

(22) Filed: Jun. 30, 2023

(30) Foreign Application Priority Data
Jul. 5, 2022 (JP) 2022-108702

(51) Int. Cl. A61B 3/10; A61B 3/12; A61B 3/00

(52) U.S. Cl. CPC A61B 3/102; A61B 3/12; A61B 3/0041

(57) ABSTRACT

An OCT apparatus including: an OCT optical system irradiating an imaging site of an examinee's eye with measurement light, the OCT optical system detecting a spectral coherence signal of the measurement light with reference light; an observing optical system irradiating the imaging site with observation light, the observing optical system acquiring a front image of the imaging site; a changing means changing the imaging site in the OCT optical system and the observing optical system between a fundus of the examinee's eye and an anterior segment; a driving part moving an imaging unit including the OCT optical system and the observing optical system with respect to the examinee's eye; and a controller. The controller displays a front image of the anterior segment sequentially acquired through the observing optical system in a first region on a screen, and receives an operation input that controls the driving part through the first region.

### CROSS-REFERENCE TO RELATED APPLICATION

This application claims priority from Japanese Patent Application No. 2022-108702 filed on Jul. 5, 2022, with the Japan Patent Office, the entire contents of which are hereby incorporated by reference.

### BACKGROUND ART

### 1. Technical Field

The present disclosure relates to an OCT apparatus.

### 2. Related Art

In the field of ophthalmology, as an optical coherence tomography (OCT) apparatus that images a tomographic image of a tissue of an examinee's eye, there is known an apparatus that selectively images fundus OCT and anterior segment OCT by switching an optical system. For example, JP-A-2014-138904 discloses an apparatus capable of simply imaging the anterior segment OCT by attaching an adapter lens to the fundus OCT.

### SUMMARY

An OCT apparatus according to the present disclosure including: an OCT optical system configured to irradiate an imaging site of an examinee's eye with measurement light, the OCT optical system being configured to detect a spectral coherence signal of the measurement light with reference light; an observing optical system configured to irradiate the imaging site with observation light, the observing optical system being configured to acquire a front image of the imaging site based on reflected light of observation light from the imaging site; a changing means configured to change the imaging site in the OCT optical system and the observing optical system between a fundus of the examinee's eye and an anterior segment; a driving part configured to move an imaging unit including the OCT optical system and the observing optical system with respect to the examinee's eye; and a controller, in which when the anterior segment of the examinee's eye is the imaging site, the controller displays a front image of the anterior segment sequentially acquired through the observing optical system in a first region on a screen, and the controller receives an operation input that controls the driving part through the first region.

### BRIEF DESCRIPTION OF DRAWINGS

FIG. 1 is a diagram illustrating an apparatus configuration of an OCT apparatus according to an embodiment;

FIG. 2 is a diagram illustrating an apparatus configuration when an anterior segment is imaged;

FIG. 3 is a flowchart illustrating a flow of operation in the embodiment;

FIG. 4 illustrates an imaging screen in a fundus mode according to the embodiment; and

FIG. 5 illustrates an imaging screen in an anterior segment mode according to the embodiment.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

However, in an apparatus capable of simply imaging the anterior segment OCT, the operability of the apparatus when imaging the anterior segment OCT has not been sufficiently studied. On the other hand, the present disclosure has been made in view of the problems of the background art, and an object of the present disclosure is to provide an OCT apparatus that facilitates the imaging of anterior segment OCT even by an inexperienced examiner.

An OCT apparatus according to a first aspect of the present invention includes: an OCT optical system configured to irradiate an imaging site of an examinee's eye with measurement light, the OCT optical system being configured to detect a spectral coherence signal of the measurement light with reference light; an observing optical system configured to irradiate the imaging site with observation light, the observing optical system being configured to acquire a front image of the imaging site based on reflected light of observation light from the imaging site; a changing means configured to change the imaging site in the OCT optical system and the observing optical system between a fundus of the examinee's eye and an anterior segment; a driving part configured to move an imaging unit including the OCT optical system and the observing optical system with respect to the examinee's eye; and a controller. When the anterior segment of the examinee's eye is the imaging site, the controller displays a front image of the anterior segment sequentially acquired through the observing optical system in a first region on a screen, and the controller receives an operation input that controls the driving part through the first region.

According to the present disclosure, it is possible to provide an OCT apparatus that is capable of easily imaging the anterior segment OCT by an inexperienced examiner.

Overview

An OCT apparatus exemplified in the present disclosure includes at least an OCT optical system, an observing optical system, a changing part, a driving part, and a controller. The OCT optical system irradiates the imaging site of the examinee's eye with the measurement light, and detects a spectral coherence signal between the measurement light and the reference light. The observing optical system is used to irradiate an imaging site with observation light and acquire a front image of the imaging site based on reflected light of the observation light from the imaging site. The changing part changes the imaging site of the examinee's eye in the OCT optical system and the observing optical system between the fundus and the anterior segment of the examinee's eye. The driving part moves the imaging unit including the OCT optical system and the observing optical system with respect to the examinee's eye. When the anterior segment of the examinee's eye is an imaging site, the controller displays the front image of the anterior segment sequentially acquired through the observing optical system in a first region on the screen. Furthermore, when the anterior segment of the examinee's eye is an imaging site, the controller receives an operation input that controls the driving part through the first region.

When the anterior segment of the examinee's eye is an imaging site, the position of the imaging unit with respect to the examinee's eye can be confirmed through the first region where the front image of the anterior segment sequentially acquired is displayed, and the operation that moves the imaging unit can be input, so that the operation of adjusting the position of the imaging unit in an anterior segment mode by the examiner becomes easy.

Furthermore, when the anterior segment of the examinee's eye is an imaging site, the controller may receive an operation input that specifies a position on the front image of the anterior segment in the first region through a pointing device. Furthermore, when the anterior segment of the examinee's eye is an imaging site, the controller may move the imaging unit with respect to the examinee's eye in the XY direction such that the tissue of the anterior segment displayed at the position specified by the pointer is displayed at a predetermined position in the first region. As a result, the optical axes of the OCT optical system and the observing optical system are moved in the XY directions.

When the pointing device is operated, the imaging unit is moved such that the tissue of the anterior segment displayed at the specified position in the first region is displayed at the predetermined position in the first region, so that it is possible to easily move the imaging unit to a desired position of the examiner. The rough scan position of the anterior segment OCT in the XY direction is determined according to the position of the imaging unit in the XY direction with respect to the examinee's eye. Therefore, the scan position in the XY direction of the anterior segment OCT is easily adjusted based on the operation input that specifies the position on the front image of the anterior segment in the first region.

However, another operation input may be adopted as the operation input through the pointing device. For example, when the front image of the anterior segment displayed in the first region is dragged by the pointing device, the driving part may be controlled such that an imaging unit 2 is moved in a direction opposite to the moving direction of the drag corresponding to the moving direction and the moving amount of the drag. Furthermore, the controller may receive an operation input through a touchscreen. The touchscreen is provided with a first region, and in the anterior segment mode, a front image of the anterior segment is displayed in the first region. In this case, the driving part may be controlled based on a flick operation, a rotation gesture operation, or a long tap operation on the first region detected by the touchscreen to adjust the position of the imaging unit 2 with respect to an examinee's eye E (for details of the operation of the driving part corresponding to the operation input and the operation input, refer to, for example, JP-A-2014-205078 and JP-A-2017-176545 by the present applicant.

When the fundus of the examinee's eye is an imaging site, the controller displays a front image of the fundus sequentially acquired through the observing optical system in the first region, and receives an operation input that changes a scan position on the fundus in the XY direction through the first region. In this case, the controller changes the scan position on the fundus in the XY direction by controlling at least one of the OCT optical system and the fixation optical system. As described above, in both when the imaging site is the anterior segment and when the imaging site is the fundus, the operation received through the first region is an operation that changes the scan position in the XY direction, so that even an inexperienced examiner can easily understand the operation method of the apparatus.

Note that the position of the first region on the screen may be constant between when the imaging site is the anterior segment and when the imaging site is the fundus. Furthermore, the screen layout other than the first region may be the same. This makes it easier for an inexperienced examiner to understand the operation method of the apparatus.

Furthermore, the OCT apparatus may include a second observing optical system different from the observing optical system. The second observing optical system is used to observe the anterior segment of the examinee's eye when the fundus of the examinee's eye is an imaging site. When the fundus of the examinee's eye is an imaging site, the controller displays the front image of the anterior segment sequentially acquired through the second observing optical system in the second region as a second observation image. As a result, when the fundus of the examinee's eye is an imaging site, the position adjustment of the imaging unit with respect to the examinee's eye becomes easy by using the observation image based on the front image of the anterior segment. Furthermore, automatic alignment and tracking may be executed based on the second observation image. Note that the observing optical system and the second observing optical system may have different light sources and photo detectors (imaging devices).

When the anterior segment of the examinee's eye is an imaging site, the controller may stop the display control of the second observation image in the second region. when the anterior segment of the examinee's eye is an imaging site, it is difficult to acquire an appropriate front image of the anterior segment by the second observing optical system. Therefore, the display control of the second observation image in the second region is stopped, and the observation image of the anterior segment acquired through the observing optical system is displayed in the first region. Accordingly, the examiner is hard to misunderstand the adjustment state of the position of the imaging unit 2 with respect to the examinee's eye E.

A third region may be disposed on the screen together with the first region. The controller may display a live image by the OCT image of the examinee's eye sequentially acquired through the OCT optical system in the third region.

### Embodiment

An embodiment of the present disclosure is illustrated with reference to the drawings. An OCT apparatus 1 according to the embodiment acquires OCT data of an examinee's eye. In the OCT apparatus 1 of the present embodiment, OCT data of a fundus and OCT data of an anterior segment are selectively imaged.

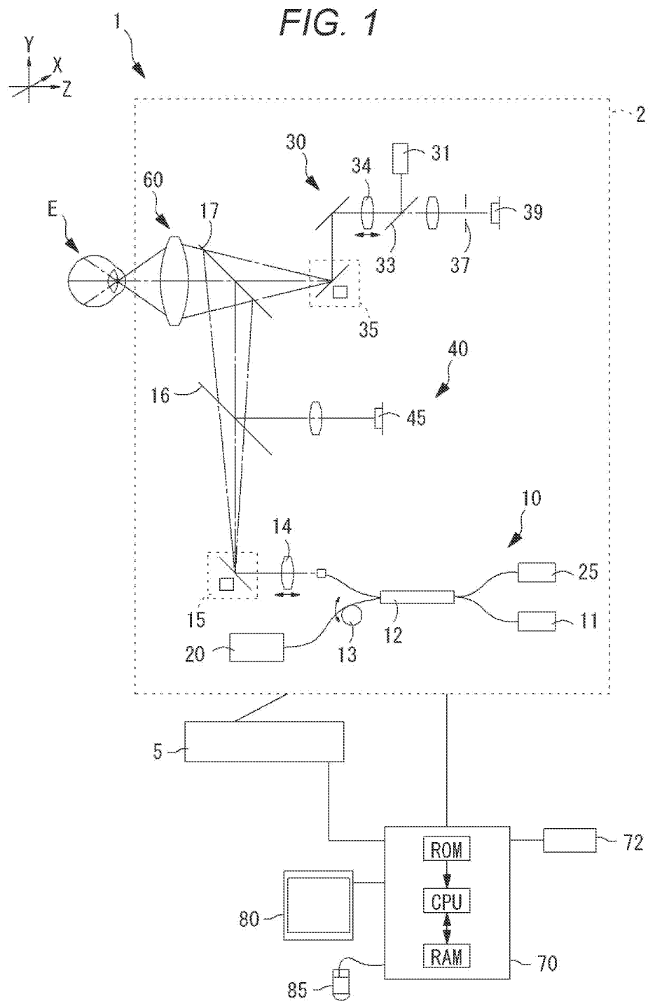

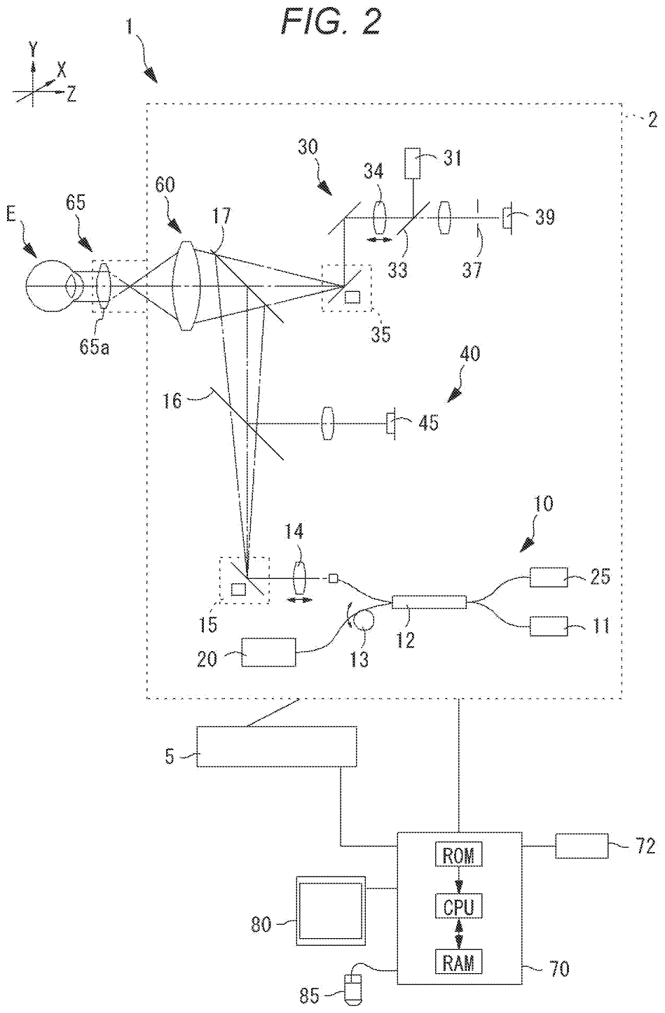

First, a configuration of the OCT apparatus 1 will be described with reference to FIGS. 1 and 2. FIG. 1 illustrates an apparatus configuration in a state where OCT data of a fundus can be imaged. FIG. 2 illustrates an apparatus configuration in a state where the OCT data of the anterior segment can be imaged. FIG. 2 is different from FIG. 1 in that an anterior segment attachment 65 is attached to an imaging unit 2.

Note that in the description of the embodiment, the axial direction of an examinee's eye E is described as a Z direction, the horizontal direction is described as an X direction, and the vertical direction is described as a Y direction.

As illustrated in FIGS. 1 and 2, the OCT apparatus 1 according to the embodiment includes the imaging unit 2, a driving part 5, and a controller 70.

Measurement Part

The imaging unit 2 has a main optical system in the OCT apparatus 1. In the present embodiment, the imaging unit 2 includes an OCT optical system (interference optical system) 10, a first observing optical system (SLO optical system) 30, and a second observing optical system 40 (anterior segment observing optical system). Optical paths of the OCT optical system 10, the first observing optical system 30, and the second observing optical system 40 are branched/coupled by beam splitters/combiners 16 and 17.

OCT Optical System

The OCT optical system 10 detects a spectral coherence signal between measurement light with which the examinee's eye is irradiated and reference light. The OCT optical system 10 may be, for example, SD-OCT, SS-OCT, or OCT based on another imaging principle.

The OCT optical system 10 includes at least an OCT light source 11, a light splitter 12, a reference light optical system 20, and a detector 25. Note that the reference light optical system 20 in the present embodiment will be described as a reflective optical system, but may be a transmissive optical system.

The OCT light source 11 emits low coherent light. The light emitted from the OCT light source 11 is divided into measurement light and reference light by the light splitter 12. In the present embodiment, a coupler (splitter) is used as the light splitter 12. The measurement light is guided to the examinee's eye through a light guiding optical system 10a, and the reference light is guided to the reference light optical system 20. In FIG. 1, a polarizer 13 is disposed on the reference optical path. The reference light is reflected by a mirror (not illustrated) disposed on the reference optical path, and enters the detector 25 in a state of being multiplexed with the return light of the measurement light by the light splitter 12. As a result, a spectral coherence signal between the return light and the reference light is detected. For example, in the SD-OCT, a spectrometer is used as the detector 25.

In the present embodiment, the mirror (not illustrated) disposed in the reference light optical system 20 is movable along the optical axis, and the optical path length difference between the measurement light and the reference light is adjusted according to the position of the mirror. Furthermore, the polarizer 13 adjusts the polarization of the measurement light and the reference light.

In addition to this, a focusing lens 14, a scanning unit (optical scanner) 15, and an objective lens 60 are disposed on an optical path between the light splitter 12 and the examinee's eye E.

In the present embodiment, the focusing lens 14 is displaced in the optical axial direction to change the focus position in the OCT optical system 10.

The scanning unit 15 is used to change the acquisition position of the OCT image. The scanning unit 15 may be used to two-dimensionally scan the imaging site of the examinee's eye with the measurement light. The scanning unit 15 may include, for example, two optical scanners having different scanning directions. The optical scanners may be a galvanometer mirror or another optical scanner.

The objective lens 60 guides the measurement light to the imaging site in the examinee's eye. The measurement light is turned about a position conjugate with the scanning unit 15 through the objective lens 60 as a pivot point. As illustrated in FIG. 1, when the anterior segment of the examinee's eye is located at the pivot point, the measurement light reaches the fundus without being eclipsed by the iris, and the measurement light is scanned on the fundus based on the drive of the scanning unit 15. In this case, the light collecting surface of the measurement light is formed on the fundus.

Anterior Segment Attachment

In the present embodiment, the anterior segment attachment 65 is used to change the imaging site in the OCT optical system 10 and the first observing optical system 30 between the fundus of the examinee's eye E and the anterior segment. As illustrated in FIG. 2, when the anterior segment OCT is acquired, the anterior segment attachment 65 (attachment lens 65a) is inserted between the objective lens 60 and the examinee's eye E. As a result, the scanning mode of the measurement light is changed between the case of acquiring the fundus OCT and the case of acquiring the anterior segment OCT. By inserting the attachment lens 65a, the measurement light is irradiated in a telecentric manner, and the anterior segment OCT is acquired. Furthermore, by inserting the attachment lens 65a, the condensing state of the measurement light is changed such that the condensing surfaces of the measurement light are positioned at the anterior segment.

First Observing Optical System

The first observing optical system 30 is used to acquire a front image of an imaging site as an observation image. In a state where the anterior segment attachment 65 is not attached (the anterior segment attachment 65 is retracted), a front image of the fundus is acquired as an observation image through the first observing optical system 30. In a state where the anterior segment attachment 65 is attached, a front image of the anterior segment is acquired as an observation image through the first observing optical system 30.

In FIG. 1, an SLO optical system is illustrated as an example of the first observing optical system 30. The first observing optical system 30 may include at least an irradiation optical system and a light receiving optical system. The irradiation optical system irradiates an imaging site of the examinee's eye with observation light. The light receiving optical system receives fundus reflected light by observation light by the photo detector 39. Observation images are sequentially acquired based on the output signal from the photo detector 30.

The first observing optical system 30 further includes a focus adjustment unit. The focus adjustment unit includes a focusing lens 34.

As the observation light source 31, for example, a laser diode light source is used. In addition to the focusing lens 34, a scanning unit 35 and an objective lens 60 are disposed in the observation optical path. The scanning unit 35 two-dimensionally scans the imaging site of the examinee's eye with light. The scanning unit 35 may include, for example, a combination of a polygon mirror and a galvano scanner.

A beam splitter 33 is disposed between the observation light source 31 and the focusing lens 34. In the transmission direction of the beam splitter 33, a confocal opening 37 and a photo detector 39 are disposed.

The observation light is reflected by the beam splitter 33 and then reaches the scanning unit 35 through the focusing lens 34. The light that has passed through the scanning unit 35 is transmitted through the beam splitter 17 and then irradiated to the imaging site of the examinee's eye through the objective lens 60.

The reflected light from the imaging site is guided back along the light projection path to the beam splitter 33. The reflected light from the imaging site is transmitted through the beam splitter 33 and further received by the photo detector 39 through the confocal opening 37. A front image of the imaging site is formed based on the light receiving signal from the photo detector 39. The formed front image may be stored in a memory 72.

Second Observing Optical System

The second observing optical system 40 is used to observe a front image (referred to as an observation image) of the anterior segment of the examinee's eye E. The second observing optical system 40 includes at least an imaging device 45. In the present embodiment, when the imaging site of the OCT optical system 10 and the first observing optical system 30 is the fundus, an image of the anterior segment is formed on the imaging device 45. That is, in a state where the anterior segment attachment 65 is not mounted, the imaging device 45 and the anterior segment have a substantially conjugate relationship. The observation image of the anterior segment acquired through the second observing optical system 40 is used for alignment of the imaging unit 2 when the examinee's eye E at the time of fundus is imaged.

Fixation Target Projecting Optical System

The OCT apparatus 1 further includes a fixation target projecting optical system. The fixation target projecting optical system may be an internal fixation light. The fixation target projecting optical system projects a fixation target (fixation light flux) onto the examinee's eye E, so that the direction of the visual line of the examinee's eye E is guided. In the present embodiment, the fixation target projecting optical system can two-dimensionally change the presented position of the fixation target and can guide the examinee's eye E in a plurality of directions. As a result, the imaging site is changed. In the present embodiment, the fixation projecting optical system is shared by the first observing optical system 30 which is an SLO optical system. By providing a visible light source different from the observation light source and controlling the projection timing of the visible light, the fixation target is projected onto the examinee's eye E.

Driving Part

The driving part 5 moves the imaging unit 3 in the XYZ directions with respect to the examinee's eye E. The driving part 5 includes an actuator that moves the imaging unit 2 in each direction, and is driven based on a control signal from the controller 70.

Control System

Next, the control system of the OCT apparatus 1 will be described.

The controller 70 of the OCT apparatus 1 controls various operations in the OCT apparatus 1. Furthermore, in the present embodiment, various types of image processing are performed by the controller 70. That is, the image processor is shared by the controller 70. The controller 70 may include, for example, a CPU, a RAM, a ROM, and the like.

Furthermore, in the present embodiment, the controller 70 is connected to a monitor 80 and performs display control of the monitor 80. Furthermore, the controller 70 is connected to the memory 72, the operating unit 85, and the like.

In the present embodiment, a pointing device such as a mouse may be provided as the operating unit 85. Furthermore, the monitor 80 may be a touchscreen display, and in this case, the monitor 80 shares the operating unit 85. The monitor 80 and the operating unit 85 may be disposed at a remote place from the OCT apparatus 1 through a network or the like.

Description of Operation

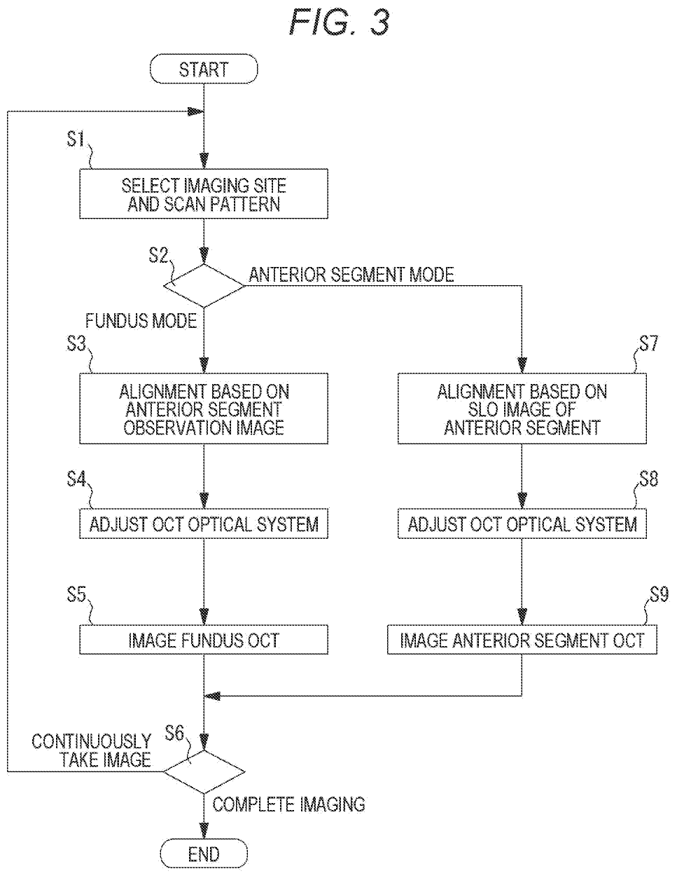

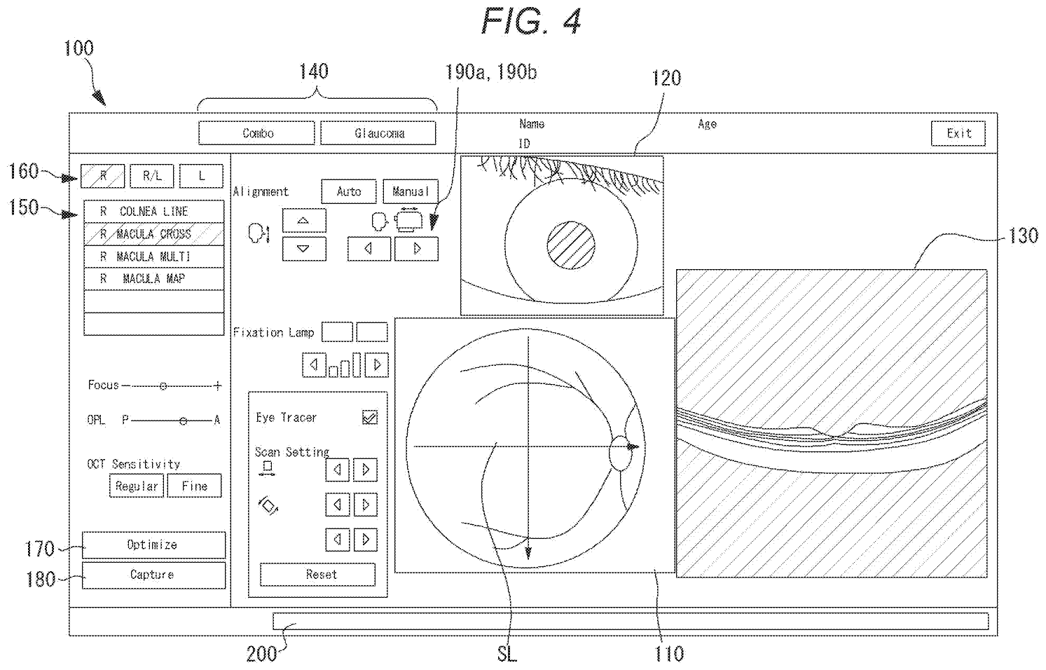

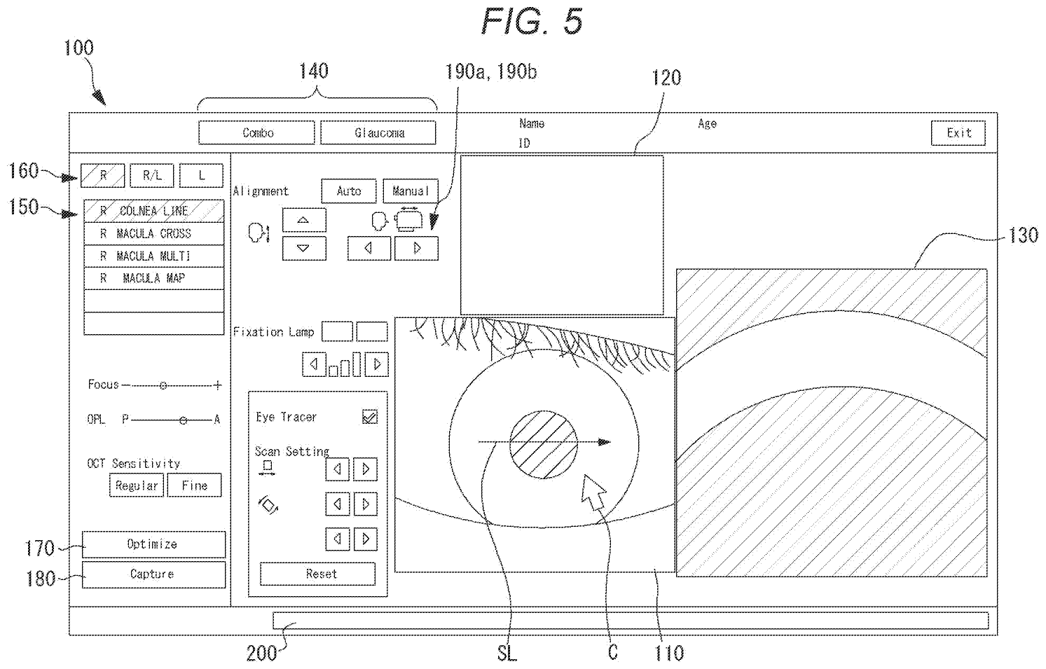

Next, an operation in the OCT apparatus 1 will be described with reference to FIGS. 3 to 5. FIG. 3 is a flowchart illustrating a flow of operation in the embodiment. Various operations at the time of imaging are input through an imaging screen 100 illustrated in FIGS. 4 and 5. FIG. 4 illustrates a screen configuration when the OCT data of the fundus is imaged. FIG. 5 illustrates a screen configuration when the OCT data of the anterior segment is imaged. On the imaging screen 100 of the present embodiment, for example, at least a first region 110, a second region 120, and a third region 130 are provided as display regions in which an image of the examinee's eye acquired through the optical system is displayed. The first region 110 is used to display the front image of the imaging site. For example, an observation image acquired through the first observing optical system 30 may be displayed in the first region 110. A second observation image acquired through the second observing optical system 40 is displayed in the second region 120. The third region 130 is used to display the OCT image of the imaging site. For example, a real-time B-scan image may be displayed in the first region 110. As illustrated in FIGS. 4 and 5, in both the case of imaging the OCT data of the fundus and the case of imaging the OCT data of the anterior segment, the disposition of the display regions 110, 120, and 130 on the imaging screen 100 is constant. Furthermore, on the imaging screen 100, various user interfaces that set imaging conditions and displaying the state of the apparatus are disposed (details will be described later).

For example, in the present embodiment, first, an imaging site and a scan pattern are set (S1). Various scan patterns such as line, cross, multi, map, radial, and circle patterns are known as OCT scan patterns. In the OCT apparatus 1 of the present embodiment, a plurality of combinations of one or more scan patterns is prepared in advance according to the disease and the application. In each combination, the imaging site is predetermined for each scan pattern. The examiner can select any combination according to the disease and the use by operating a scan pattern selection unit 140.

The breakdown of each scan pattern included in the selected combination is reflected in the content of a list 150. In the list 150 of FIGS. 4 and 5, text indicating scan settings for each scan is displayed. The text includes, as scan settings, an imaging target eye (any one of left or right), an imaging site, and a scan pattern. The imaging target eye can be selected and changed by operating an imaging target eye selection button 160.

Imaging is sequentially performed in accordance with the scan settings displayed in the list 150. Normally, imaging is performed in order from the top to the bottom of the list 150. When a scan setting in the next imaging (executed when a capture button 180 is subsequently operated) is selected, the text corresponding to the selected scan setting is highlighted on the list 150. Furthermore, by performing a selection operation with a pointing device on the text corresponding to the desired scan setting of the examiner, the scan setting in the next imaging can be changed.

Fundus Mode

When the scan setting for fundus imaging is selected, the imaging mode of the apparatus is set to the fundus mode by the controller 70 (S2: fundus mode). In this case, the examiner retracts the anterior segment attachment 65.

In the fundus mode, the controller 70 controls the first observing optical system and the second observing optical system 40 to acquire an observation image and display the observation image on the imaging screen 100 (see FIG. 4).

After that, alignment is performed automatically or manually (S3). The position of the imaging unit 2 with respect to the examinee's eye E is adjusted based on the observation image of the anterior segment displayed in the second region 120. In adjusting the position of the imaging unit 2, an alignment target may be projected on the cornea of the examinee's eye, and a target image reflected in the anterior segment observation image may be used (for details, see, for example, JP-A-2015-195874 by the present applicant.). As a result of the alignment, in regard to the XY direction, the optical axes of the OCT optical system 10 and the first observing optical system 30 are adjusted to pass through the pupil. As a result, an observation image of the fundus acquired through the first observing optical system 30 is displayed in the first region 110. In regard to the Z direction, the operation distance from the examinee's eye E to the imaging unit 2 is adjusted to a predetermined value (allowable range).

At least when the alignment is completed, the controller 70 controls the OCT optical system 10 to start acquisition of OCT data of the examinee's eye E.

In the present embodiment, after the alignment is completed, the examiner operates an optimize button 170 to execute adjustment processing of the OCT optical system 10 (S4). In the adjustment processing of the present embodiment, as an example, the optical path length difference, the focus, and the polarization are adjusted. By adjusting the optical path length difference according to the ocular axial length of the examinee's eye, the tomographic image of the fundus is displayed in the third region 130. By adjusting the focus and the polarization, a tomographic image can be imaged with good image quality.

In the present embodiment, a graphic (in the following, it is referred to as a scan line SL) indicating a scan position (scan line) is superposed on the fundus observation image of the first region 110. The scan line SL is superposed at the scan position corresponding to the selected scan setting (scan pattern). In the present embodiment, an operation of moving the position of the scan line SL may be inputtable on the first region 110, and the scan position on the fundus may be changeable based on the operation. As the operation of moving the position of the scan line SL, any one of parallel movement, rotational movement, movement of an end point, and movement of an intersection (of a plurality of scan lines) of the scan line SL may be used.

For example, the presented position of the fixation target may be changeable through the fundus observation image of the first region 110. For example, when the examiner selects one point on the fundus observation image, the presented position of the fixation target may be controlled such that the one point is located at the center of the image in the first region 110.

When the capture button 180 is operated, an OCT image is imaged at a preset scan position (S5). The imaging result may be displayed on the monitor 80 every time when the examiner confirms the imaging result. When the imaging is successful, the image is stored in the memory 72, and when the imaging is unsuccessful, the imaging may be performed again.

In the present embodiment, when imaging for any of the scan settings shown in the list 150 is not completed, the process returns to S1 and continues. Note that at this time, the combination of scan patterns may be taken over, and one of the scan settings shown in the list 150 before imaging may be automatically selected as the next scan setting. On the other hand, in the present embodiment, when imaging has been completed for all the scan settings shown in the list 150, a series of imaging operations is completed (S6: complete imaging).

Anterior Segment Mode

The anterior segment mode will be described. When the scan setting for fundus mode is selected, the imaging mode of the apparatus is set to the anterior segment mode by the controller 70 (S2: anterior segment mode). In this case, the examiner wears the anterior segment attachment 65. In the anterior segment mode of the present embodiment, the position of the focus lens 14 in the OCT optical system 10 and the optical path length difference between the measurement light and the reference light (the position of the mirror, not illustrated, in the reference light optical system 20) are adjusted to predetermined positions. Furthermore, the same applies to the position of the focus lens 34 in the first observing optical system 30. After that, in the anterior segment mode, the movement of the position of the focus lens 14 in the OCT optical system 10, the optical path length difference between the measurement light and the reference light, and the position of the focus lens 34 in the first observing optical system 30 is limited. For details, see, for example, JP-A-2011-147609 by the present applicant.

In the anterior segment mode, the controller 70 controls the first observing optical system 30 to acquire the front image of the anterior segment as an observation image and display the front image on the imaging screen 100 (see FIG. 5). Furthermore, the controller 70 controls the OCT optical system 10 to start acquisition of OCT data. The OCT data acquired as needed is displayed in the third region 130.

On the other hand, in the anterior segment mode, the second observing optical system 40 is out of focus on the anterior segment. When the anterior segment front image that is out of focus with respect to the second region 120 is displayed in the anterior segment mode, even though the position of the imaging unit 2 with respect to the examinee's eye E is appropriately adjusted, the examiner may misunderstand that the position of the imaging unit 2 is in an inappropriate state. Therefore, the controller 70 controls the second observing optical system 40 and the anterior segment illumination so as not to acquire the second observation image through the second observing optical system 40, and further performs display control on the second region 120.

After that, alignment is performed automatically or manually (S7). The position of the imaging unit 2 with respect to the examinee's eye E is adjusted based on the observation image of the anterior segment displayed in the first region 110.

Here, a manual alignment method will be exemplified. An operation input that moves the imaging unit 2 is received through the first region 110 (and an observation image of the anterior segment displayed in the first region 110.). For example, in regard to the XY direction, when a position is specified on the observation image of the anterior segment through the pointing device, the imaging unit 2 moves with respect to the examinee's eye E such that the tissue of the anterior segment displayed at the specified position is displayed at a predetermined position in the first region 110.

In the present embodiment, the predetermined position in the first region 110 is assumed to be at the center of the first region 110. However, the present embodiment is not necessarily limited to this, and other positions can be appropriately adopted as the predetermined position.

In regard to the Z direction, first, the imaging unit 2 is moved roughly in a direction in which the observation image is focused while referring to the observation image displayed in the first region 110. When the OCT data of the anterior segment is depicted in the third region 130 as a result of the movement, the position of the imaging unit 2 is adjusted in the Z direction such that the image of the desired depth region in the anterior segment is depicted in the third region 130.

In order to move the imaging unit 2 in the Z direction, the controller 70 is required to perform an operation different from the operation that moves the imaging unit 2 in the XY direction. For example, in FIGS. 4 and 5, the imaging unit 2 is moved in the Z direction by operating forward and backward movement buttons 190a and 190b. The forward and backward movement buttons 190a and 190b are two buttons corresponding to forward movement and backward movement, respectively. The controller 70 continuously moves the imaging unit 2 in a direction corresponding to the pressed button. In this case, the movement amount of the imaging unit 2 is proportional to the pressing time of the button. However, the operation that moves the imaging unit 2 in the Z direction is not necessarily limited to this, and various other operations can be used in combination or substituted. For example, when a mouse is used as the pointing device, a wheel operation of the mouse may be used. In this case, when the cursor C (pointer) on the imaging screen 100 is disposed in the first region 110, the controller 70 may move the imaging unit 2 in the Z direction by inputting the wheel operation. The movement amount of the imaging unit 2 may be determined according to the operation amount of the wheel. Furthermore, when the monitor 80 is a touchscreen display, pinch-in/pinch-out on the first region 110 may be used.

Note that in the anterior segment mode of the present embodiment, as a result of attaching the anterior segment attachment 65, the operation distance is shorter than that in the fundus mode. On the other hand, the driving part 5 may be controlled such that the moving speed of the imaging unit 3 in the anterior segment mode is slower than that in the fundus mode. Furthermore, when the movement amount of the imaging unit 3 is determined according to the input amount (operation amount) as in the operation for the movement in the Z direction of the present embodiment, the driving part 5 may be controlled such that the movement amount with respect to the input amount (operation amount) in the anterior segment mode is smaller than that in the fundus mode.

As described above, in the anterior segment mode, the position of the imaging unit 2 with respect to the examinee's eye E can be confirmed through the first region 110, and an operation that moves the imaging unit 2 can be input, so that the operation of adjusting the position of the imaging unit 2 in the anterior segment mode by the examiner becomes easy. Furthermore, although the second observing optical system 40 is out of focus in the anterior segment mode, the anterior segment front image is not displayed in the second region 120 in the anterior segment mode. Therefore, the examiner is hard to misunderstand the adjustment state of the position of the imaging unit 2 with respect to the examinee's eye E.

In the present embodiment, a graphic (in the following, it is referred to as a scan line SL) indicating a scan position (scan line) is superposed on the anterior segment observation image of the first region 110. An operation of moving the position of the scan line SL may be inputtable on the first region 110, and the scan position on the anterior segment may be changeable based on the operation.

After the desired scan position is set, an OCT image of the anterior segment is imaged by operating the capture button 180 (S8).

Modifications

Although the present disclosure has been described above based on the embodiment, the present disclosure is not limited to the foregoing embodiments, and various modifications can be made.

For example, in the foregoing embodiment, as an example of a changing means that changes the imaging site in the OCT optical system 10 and the first observing optical system 30 between the fundus of the examinee's eye E and the anterior segment, one that changes the imaging site by attaching and detaching the anterior segment attachment 65 (and the attachment lens 65a) and switching the objective optical system is exemplified, but the changing means that changes the imaging site is not necessarily limited to the changing means that switches the objective optical system. For example, the imaging site in the OCT optical system and the first observing optical system may be changed by changing at least the interval between the examinee's eye and the imaging unit without changing the objective optical system between the case of imaging the fundus and the case of imaging the anterior segment. For example, when the fundus is imaged, it is possible to change the imaging site to the anterior segment by moving the imaging unit away from the examinee's eye. In this case, the changing means may be, for example, a driving part that moves the imaging unit, or a mechanism that changes the position of a face support unit (not illustrated) that supports the face of the examinee. Since it is difficult to acquire an appropriate observation image through the second observing optical system by changing the interval between the examinee's eye and the imaging unit, the operation input and control through the first region when imaging the anterior segment OCT are useful similarly to the foregoing embodiment.

Furthermore, for example, when the OCT of the anterior segment is imaged, an operation that adjusts the position of the imaging unit 2 with respect to the examinee's eye E may be received through the OCT image of the anterior segment displayed in the third display region 130. For example, when a position is specified on the tomographic image through the pointing device, the imaging unit 2 may move with respect to the examinee's eye E such that the tissue of the anterior segment displayed at the specified position is displayed at a predetermined position in the third region 130 and the first region 110. In this case, the OCT image of the anterior segment may be moved in a direction along the scan line (transverse direction) or may be moved in the depth direction.

Furthermore, in the foregoing embodiment, since the anterior segment is imaged by the substantially telecentric optical system on the object side by attaching the anterior segment attachment 60, the inclination of the OCT image does not change so much even though the positional relationship between the examinee's eye E and the imaging unit 2 is changed with respect to the XY direction. However, in acquiring the OCT of the anterior segment, when the optical system is non-telecentric on the object side, the inclination of the OCT image changes even though the positional relationship between the examinee's eye E and the imaging unit 2 is changed with respect to the XY direction. Therefore, for example, an operation input may be received to change the inclination of the OCT image of the anterior segment displayed in the third display region 130, and the position of the imaging unit 2 may be controlled in the XY direction according to the operation input. For example, when a touchscreen display is used as the operating unit 85, a target inclination may be input as a rotation amount by touching and rotating two points on the OCT image.

In the foregoing embodiment, the controller 70 may detect the corneal apex in the first observation image acquired in the anterior segment mode. The corneal apex may be detected, for example, based on a corneal reflection image generated in the first observation image. The corneal reflection image may be generated, for example, by reflection of observation light by a corneal apex. The controller 70 may execute guidance processing that guides the detected corneal apex to a predetermined position. In the guidance processing, for example, a graphic prompting an operation input that moves the corneal apex to a predetermined position may be displayed on the first observation image. The mode of the graphic can be appropriately set according to the operation input and the content of the drive control for the operation input. For example, after the position is specified on the observation image of the anterior segment, when the imaging unit 2 is moved with respect to the examinee's eye E such that the tissue of the anterior segment displayed at the specified position is displayed at a predetermined position in the first region 110, for example, an electronic target may be merged with respect to the detected corneal apex and displayed on the first observation image. This makes it easy to accurately specify the corneal apex on the observation image of the anterior segment. Note that the shape of the electronic target can be appropriately selected.

Furthermore, in the foregoing embodiment, when the operation distance becomes shorter than that in the fundus mode as a result of attaching the anterior segment attachment 65, the movement of the imaging unit 2 in the direction of approaching the examinee's eye E may be restricted based on the OCT image of the anterior segment of the third region. In this case, for example, when the image of the anterior segment is generated in the region on the zero delay side with respect to the predetermined threshold value in the OCT image, the controller 70 may control the driving part 5 so that the imaging unit 2 does not approach the examinee's eye E any more. Furthermore, whether an image of a specific tissue of the anterior segment on the fundus side of the cornea is generated in the OCT image is detected, and when the image of the specific tissue is generated, the driving part 5 may be controlled such that the imaging unit 2 does not approach the examinee's eye E any more. The specific tissue may be, for example, any of a crystalline lens, an iris, an anterior chamber angle, or the like, or may be another tissue. Furthermore, the present embodiment is not necessarily limited to this, and as long as the distance between the examinee's eye and the imaging unit 2 can be detected by another method, it is possible to suppress the excessive approach of the imaging unit 2 to the examinee's eye based on the detected distance.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. An OCT apparatus comprising:
an OCT optical system configured to irradiate an imaging site of an examinee's eye with measurement light, the OCT optical system being configured to detect a spectral coherence signal of the measurement light with reference light;
an observing optical system configured to irradiate the imaging site with observation light, the observing optical system being configured to acquire a front image of the imaging site based on reflected light of observation light from the imaging site;
a changing means configured to change the imaging site in the OCT optical system and the observing optical system between a fundus of the examinee's eye and an anterior segment;
a driving part configured to move an imaging unit including the OCT optical system and the observing optical system with respect to the examinee's eye; and
a controller, wherein
when the anterior segment of the examinee's eye is the imaging site, the controller displays a front image of the anterior segment sequentially acquired through the observing optical system in a first region on a screen, and the controller receives an operation input that controls the driving part through the first region.

2. The OCT apparatus according to claim 1, wherein
when the anterior segment of the examinee's eye is the imaging site, the controller receives an operation input that specifies a position on the front image of the anterior segment in the first region through a pointing device, and the controller moves the imaging unit in an XY direction with respect to the examinee's eye such that tissue of the anterior segment displayed at the specified position is displayed at a predetermined position in the first region.

3. The OCT apparatus according to claim 1, wherein
when the fundus of the examinee's eye is the imaging site, the controller displays a front image of the fundus sequentially acquired through the observing optical system in the first region, and the controller receives an operation input that changes a scan position on the fundus in an XY direction through the first region.

4. The OCT apparatus according to claim 1, comprising
a second observing optical system different from the observing optical system, the second observing optical system being configured to observe the anterior segment of the examinee's eye when the fundus of the examinee's eye is the imaging site, wherein
when the fundus of the examinee's eye is the imaging site, the controller displays, in the second region, the front image of the anterior segment sequentially acquired through the second observing optical system as a second observation image.

5. The OCT apparatus according to claim 4, wherein
when the anterior segment of the examinee's eye is the imaging site, the controller stops display control of the second observation image in the second region.

6. The OCT apparatus according to claim 1, wherein
on the screen, a third region is disposed together with the first region, and
the controller displays, in the third region, a live image by an OCT image of the examinee's eye sequentially acquired through the OCT optical system.
